(19) United States
(12) Patent Application Publication
(10) Pub. No.: US 2024/0041823 A1
(43) Pub. Date: Feb. 8, 2024

(54) USE OF MYCOLACTONE (MLN) AND DERIVATIVES THEREOF FOR TREATMENT OF MICROBIAL INFECTIONS

(71) Applicants: MICHAEL DAVID WILSON, ACCRA (GH); SAMUEL KOJO KWOFIE, ACCRA (GH); SETH OSEI ASIEDU, ACCRA (GH); PRAKASHA KEMPAIAH, NAPERVILLE, IL (US); YASH GUPTA, JACKSONVILLE, FL (US); RAVINDRA V. DURVASULA, JACKSONVILLE BEACH, FL (US)

(72) Inventors: MICHAEL DAVID WILSON, ACCRA (GH); SAMUEL KOJO KWOFIE, ACCRA (GH); SETH OSEI ASIEDU, ACCRA (GH); PRAKASHA KEMPAIAH, NAPERVILLE, IL (US); YASH GUPTA, JACKSONVILLE, FL (US); RAVINDRA V. DURVASULA, JACKSONVILLE BEACH, FL (US)

(21) Appl. No.: 18/345,605

(22) Filed: Jun. 30, 2023

Related U.S. Application Data
(60) Provisional application No. 63/369,100, filed on Jul. 22, 2022.

(51) Int. Cl. A61K 31/365; A61P 31/14

(52) U.S. Cl. CPC A61K 31/365; A61P 31/14

(57) ABSTRACT

A method of treating a microbial infection in a subject comprises administering to a subject in need thereof an effective amount of a composition comprising mycolactone (MLN). A method of preventing a microbial infection in a subject comprises administering to a subject in need thereof an effective amount of a composition comprising mycolactone (MLN).

This application claims priority from U.S. Provisional App. No. 63/369,100, filed Jul. 22, 2022, which is incorporated herein by reference.

### FIELD

The present application is directed to treatment and prevention of microbial infections, and in particular, to mycolactone (MLN) compositions and their use for treatments of microbial infection such as viral infections.

### BACKGROUND

There are few, if any, FDA-approved drugs for the prevention and/or treatment of many viral infections of public health importance, as exemplified by current COVID-19 pandemic, and outbreaks of Dengue, Yellow fever, Zika, and Ebola epidemics. The treatment approaches are mainly directed towards controlling the symptoms. The current COVID-19 pandemic has unleashed global efforts for control and treatment, which have led to vaccines and drugs, such as remdesivir and dexamethasone, as well as immunomodulatory drugs for emergency treatment. Recently, Paxlovid and molnupiravir from Pfizer and Merck/Ridgeback Biotherapeutics respectively, are authorized pills approved by the FDA. However, these drugs specifically are to prevent severe symptoms, hospitalization, and death. Paxlovid was found to be 89% effective when taken within the first three days of symptoms and 88% effective in the first five days for those considered at high risk of serious illness. Molnupiravir is less effective than anticipated and the two pills are not effective or indicated for everyone.

### SUMMARY

The present application relates to a method of preventing or treating a microbial infection in a subject. The method comprises administering to a subject in need thereof an effective amount of a composition comprising mycolactone (MLN).

One aspect of the application is a method of treating a microbial infection in a subject, comprising administering to a subject in need thereof an effective amount of a composition comprising mycolactone (MLN).

Another aspect of the application is a method of preventing a microbial infection in a subject, comprising administering to a subject in need thereof an effective amount of a composition comprising mycolactone (MLN).

In some embodiments, the subject is infected with a coronavirus, such as SARS-CoV-2.

In some embodiments, the subject is infected with a flavivirus such as Zika virus, Dengue fever virus, or yellow fever virus.

In some embodiments, the subject is infected with a filovirus, such as Ebola virus.

In some embodiments, the subject is infected with a bacterium or a fungus.

### BRIEF DESCRIPTION OF THE DRAWINGS

FIG. 1 shows binding mode and Ligplot+characterization of the MLN-CTD complex. FIG. 1(a) Docking pose of MLN in one of the binding pockets of CTD. FIG. 1(b) Two-dimensional representation of the MLN-CTD protein-ligand complex. The ball and stick model of MLN compound structure has covalent bound elements depicted for different elements i.e., Carbon as black, Oxygen red, Nitrogen blue and hydrogen as green when not bound to carbon, carbon bound hydrogen not shown to avoid confusion. MLN formed seven hydrogen bonding and fourteen hydrophobic contacts.

FIG. 2 shows graphical representations of Rg, RMSD, and RMSF of the MLN-CTD complex over a 100 ns MD simulation. FIG. 2(a) Graph of backbone RMSD (in nanometers (nm)) versus (time in nanoseconds (ps)). FIG. 2(b) Graph of RMSF (in nanometers (nm)) of the complex versus several residues. FIG. 2(c) The radius of the gyration graph of the complex (in nanometers (nm)) versus (time in picoseconds (ps)).

FIG. 3 shows FIG. 3(a) binding pocket in inactive HR1. The viral fusion domain activation involves the HR2 binding domain on HR1 to rearrange and pair with the HR2 region. FIG. 3(b) MLN A/B binding first stabilizes HR1 and second blocks the HR2 binding domain, preventing VFD activation.

FIG. 4 shows a complete spike protein and MLN A/B binding pose to Heptad repeat 1 region of the S2 domain. Heptad repeats 2 region is absent in all crystalized structures as its presence destabilizes the structure.

FIG. 5 shows the inhibition curve of MLN (hexagon data points) vs. Remdesivir (Triangular data points) against different lineages (VOCs) of SARS-CoV-2. FIG. 5(A) Alpha strain, FIG. 5(B) Delta strain, and FIG. 5(C) Omicron strain. MLN has consistent efficacy against all three VOCs.

FIG. 6 shows evaluation of antiviral activities of MLN. It shows the results of entry vs. spread assays. Against virus attachment and entry/fusion, the assay was pre-treated with MLN 2 h before adding the virus to the host cell line. The experimental procedure, virus concentration (PFU/well or MOI), and the time of addition and treatment with the test compounds are presented in the method sections. Against virus replication, the same assay was conducted with a 2 h delayed drug treatment, i.e., post-introduction of viruses giving plenty of time for the viral entry. MLN has consistent activity in both assay modes. There could be multiple mechanisms complementing as this compound completely blocks the SARS-CoV-2 cycle in both assay formats.

FIG. 7 shows MLN cytotoxicity/cytostatic effect against different cell lines at maximum concentration tested (1.34 MLN exhibited high variability in toxicity among the different cell lines. [A] Cy-totoxicity (CC50 value) against Human alveolar cell line A549 was 17.12±9.1%. [B] Cytotoxicity against immortalized Human fetal renal cell line HEK293 was 40.30±3.6%. [C] Cytotoxicity against Human hepatoma cell line Huh7.1 was 36.25±5.6%. This makes the cytotoxicity IC50 breakpoint ratio versus anti-SARS-CoV-2 activity more than 65×.

### DETAILED DESCRIPTION

The aspects of the application are described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### Definitions

In the specification and in the claims, the terms “including” and “comprising” are open-ended terms and should be interpreted to mean “including, but not limited to. . . . ” These terms encompass the more restrictive terms “consisting essentially of” and “consisting of.”

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

It must be noted that as used herein and in the appended claims, the singular forms “a,” “an,” and “the” include plural reference unless the context clearly dictates otherwise. Further, the terms “a” (or “an”), “one or more” and “at least one” can be used interchangeably herein. It is also to be noted that the terms “comprising,” “including,” “characterized by” and “having” can be used interchangeably. Further, any reactant concentrations described herein should be considered as being described on a weight to weight (w/w) basis, unless otherwise specified to the contrary (e.g., mole to mole, weight to volume (w/v), etc.).

Unless defined otherwise, all technical and scientific terms used herein have the same meanings as commonly understood by one of ordinary skill in the art to which this application belongs. All publications and patents specifically mentioned herein are incorporated by reference in their entirety for all purposes including describing and disclosing the chemicals, instruments, statistical analyses, and methodologies which are reported in the publications which might be used in connection with the application. All references cited in this specification are to be taken as indicative of the level of skill in the art. Nothing herein is to be construed as an admission that the application is not entitled to antedate such disclosure by virtue of prior invention.

The terms “mycolactone” and “MLN” are used with reference to any one of the polyketide-derived macrolide toxins from Mycobacterium species, especially Mycobacterium ulcerans, and includes but is not limited to mycolactones A, B, C, D, E, F, and dia-F, as well as stereoisomers and derivatives thereof (Kishi, 2014). Under standard laboratory conditions, mycolactones A and B exist as a 3:2 equilibrating mixture and may be referred to as mycolactone A/B.

The term “MLN composition” and “pharmaceutical composition” are used herein with reference to a composition comprising MLN and at least one pharmaceutically acceptable carrier. When referring to these compositions with regard to dosages, the weights are given in terms of the amount by weight of MLN.

The term “subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject suspected of having” means a subject exhibiting one or more clinical indicators of a microbial disease or condition.

A “subject in need thereof” means a subject identified as in need of a therapy or treatment.

A “therapeutic effect” relieves, to some extent, one or more of the symptoms of a microbial disease or disorder. “Curing” means that the symptoms of active disease are eliminated. However, certain long-term or permanent effects of the disease may exist even after a cure is obtained (such as tissue damage and the like).

The phrase “therapeutically effective amount” as used herein refers to an amount of mycolactone that ameliorates, attenuates, or eliminates one or more of the symptoms of a particular disease or condition or prevents, modifies, or delays the onset of one or more of the symptoms of a microbial disease or condition.

“Treat”, “treatment,” and “treating,” as used herein, refer to administering an MLN composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a microbial condition or disease for a period, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Administering” means providing a pharmaceutical agent or composition to a subject, and includes, but is not limited to, administering by a medical professional and self-administering.

Administration of the MLN compositions of the present application can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

“Pharmaceutical composition” means a mixture of substances suitable for administering to an individual that includes a pharmaceutical agent. For example, a pharmaceutical composition may comprise a modified oligonucleotide and a sterile aqueous solution.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable carrier” or “pharmaceutically acceptable excipient” includes all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form, however, does not imply that the dosage form is administered once per day or once per course of therapy.

The approach herein of targeting the host's physiological pathways, which are conserved whereby mutations will be deleterious, provides an effective approach for drug design, but comes with the proviso that the identified compounds are safe.

MLN binds strongly to proteins associated with exocytosis and the degranulation in platelets and mast cells, thereby blocking the initiation of cascading processes of wound healing to explain the painless feature of ulcers in M. ulcerans infections.

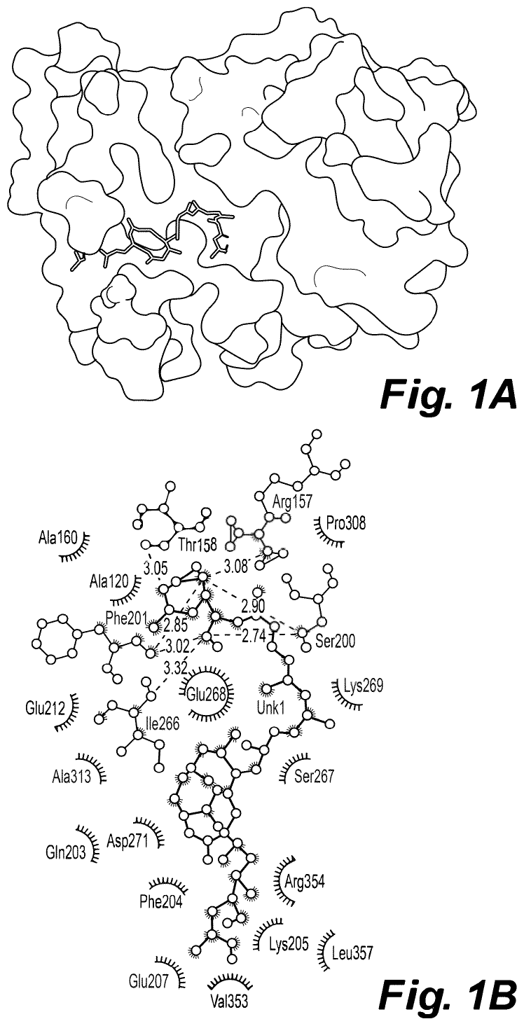

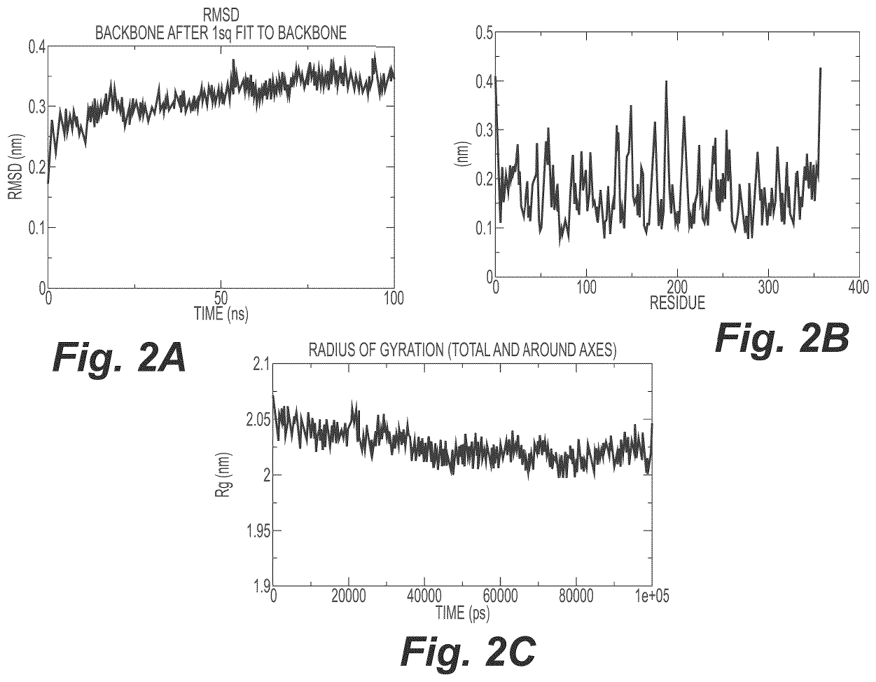

This application shows that MLN is involved in endocytosis by interacting strongly with the clathrin N terminal domain, a necessary stage of cell entry by SARS-CoV-2. The study found that MLN docked firmly inside a CTD protein binding pocket with a low binding energy of −9.0 kcal/mol and that the interactions resulted from 14 hydrophobic and five hydrogen bonds (FIG. 1). A 100 ns MD simulation revealed that the protein maintained an average RMSD of 0.3 nm, suggesting a stable structure with the Rg decreasing during the first 40 ns but remained stable for the rest of the 100 ns simulation time (FIG. 2). Using MM-PBSA calculations, the estimated average free binding energies of the complexes was −59.210 kJ/mol for the MLN-CTD complex, and the energies contributed by the electrostatic, polar, non-polar, and Van der Waals forces were −7.388 kJ/mol, 31.705 kJ/mol, −7.463 kJ/mol, and −76.177 kJ/mol, respectively, with Met99 contributing a significant −5.2131 kJ/mol.

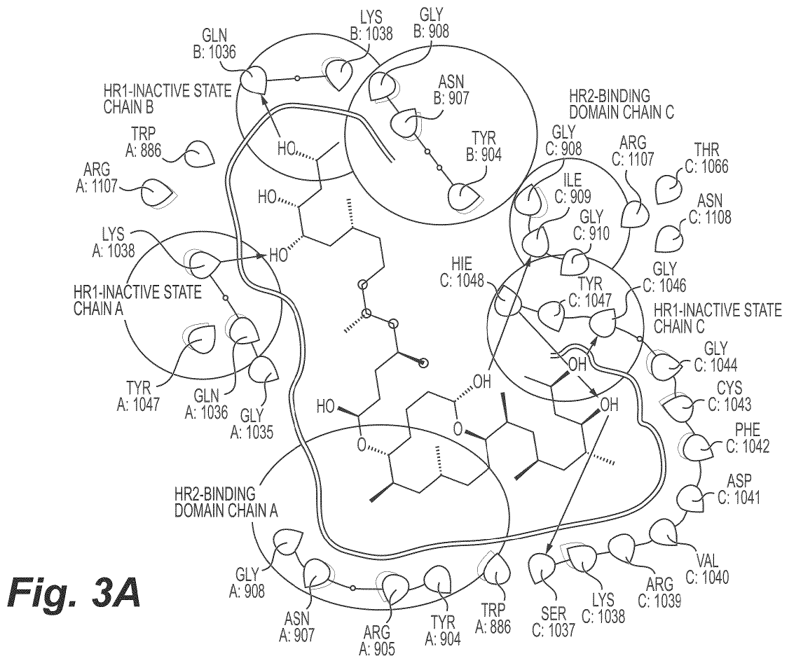

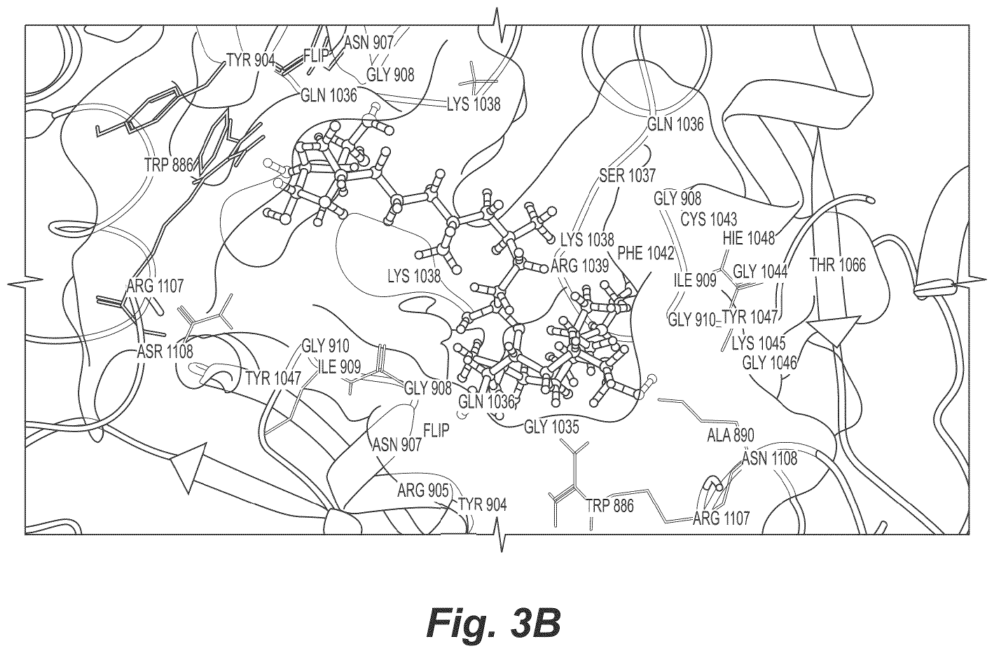

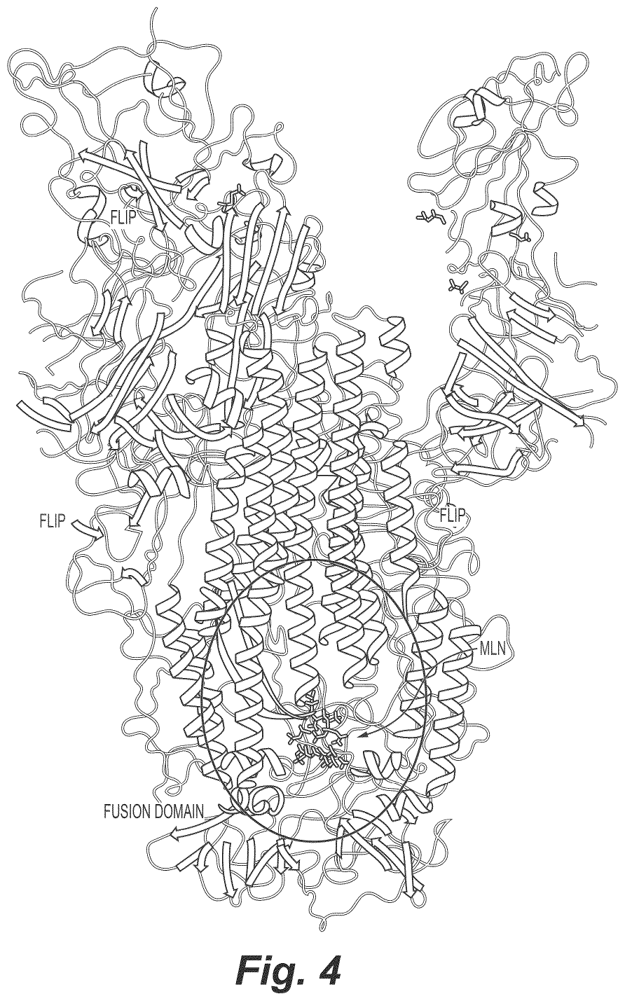

This study demonstrates that MLN is eclectic; thus, in addition to binding to the SNARE proteins involved in exocytosis of the platelets and mast cells via its interaction with Munc18b, endocytosis via binding to the clathrin-mediated pathway also has other effects on protein transports in cells by targeting sec61, which interferes with protein transport to the endoplasmic reticulum in eukaryotes and exocytosis in prokaryotes and the Sec61-dependent anti-inflammatory activity on the immune and nervous systems. It is also a ligand that mediates K+-dependent hyperpolarization through AT2R activation. Furthermore, these studies have shown that it also binds to a novel virus fusion protein of the virus. Using the reverse target screening method, this study also found a novel fusion protein that both MLN-A and MLN-B bind specifically to the HR1 region of the virus spike protein (FIG. 3a,b). HR1 region forms a dimer with HR2 during viral envelope fusion with the target cell or endosome membrane. The binding free energies of the MLN-Spike complex at the HR1 region were very high, with an average global dG bind of −62.518, and the ligand-protein complex was also highly stable throughout 20 ns MD simulations (FIG. 4). The cumulative multitarget activity not only makes this agent highly effective it also dramatically decreases the probability of resistance.

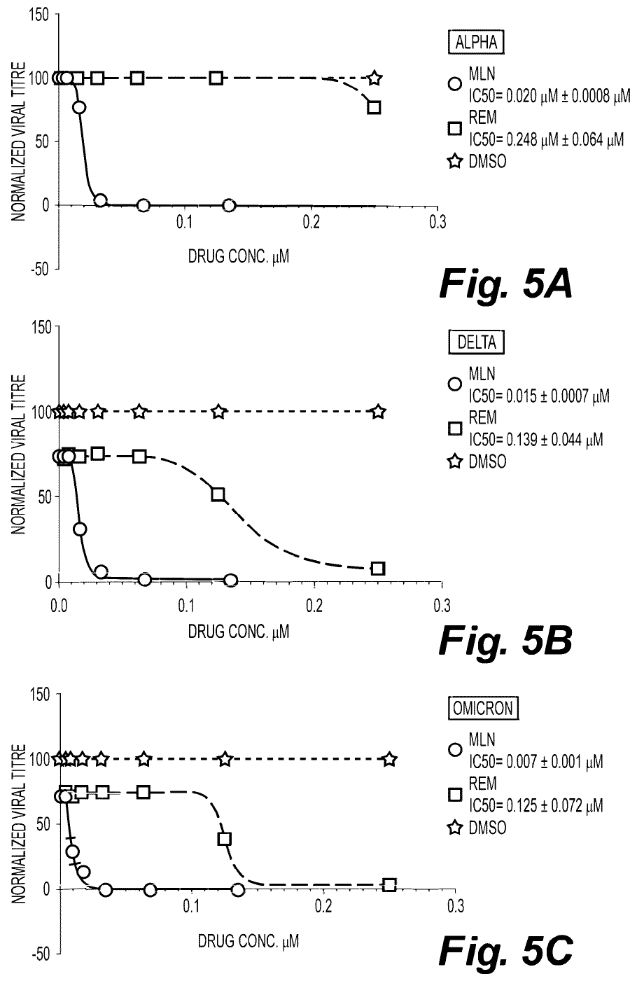

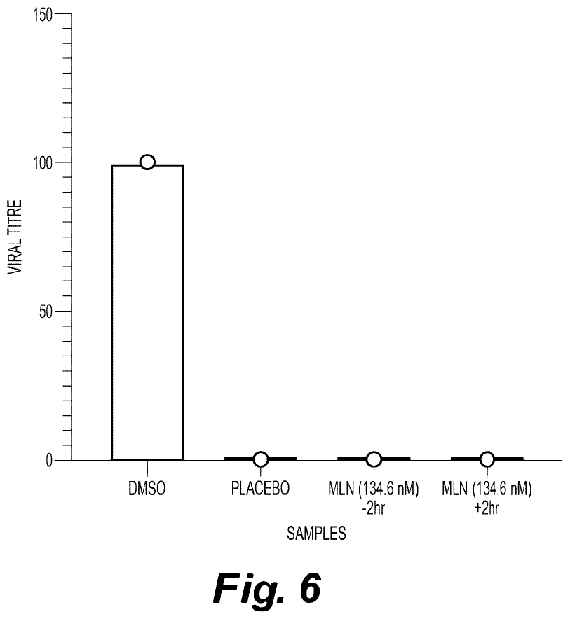

This application shows that MLN achieves 90% inhibition at 20 nM and 100% inhibition at 42 nM. When the study compared the inhibition of MLN with those of currently FDA-approved drugs Molnupiravir and PF-00835231, the 80% inhibition achieved was 0.03 μM, 0.30 μM and 0.68 μM, respectively (FIGS. 5 and 6). The study went further to test the efficacies (viral titer reduction) of MLN and remdesivir against the alpha, delta, and Omicron strains of the virus and found MLN was still comparably efficacious at 0.02 μM, 0.015 μM, and 0.007 μM compared to 0.248 μM, 0.139 μM, and 0.125 μM for remdesivir (FIG. 5). Furthermore, MLN also exhibited complete prevention of entry and spread of the virus. It is worth noting that although MLN also binds to sec16, so far, none of the numerous sec16 inhibitors that have been reported is active in the therapeutic range of >20 times their toxicities.

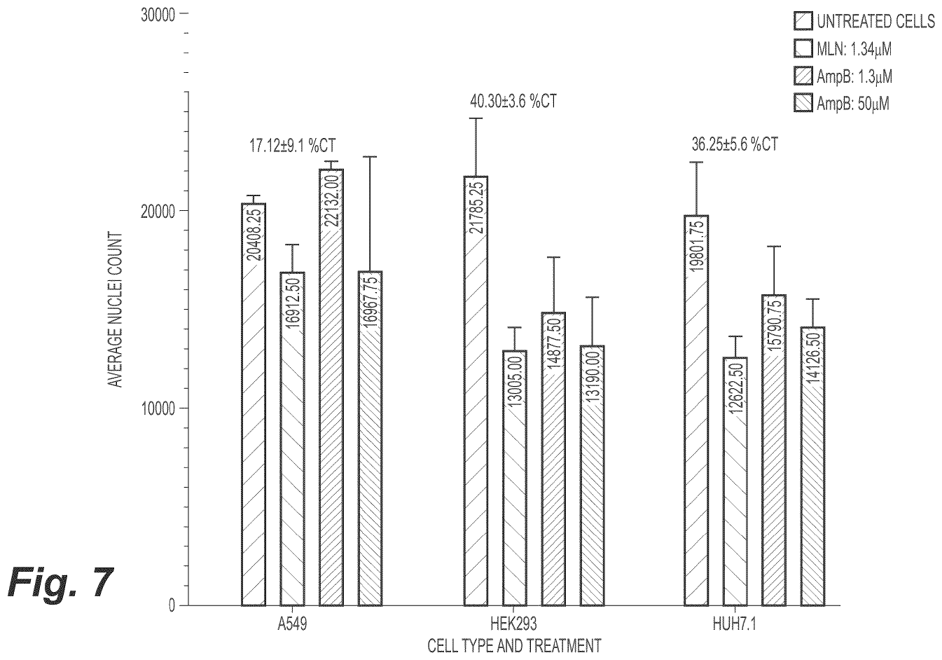

With regards to safety, the in vitro studies against human cell lines demonstrate that MLN is not cytotoxic (FIG. 7), and have demonstrated that upon intradermal injection of MLN, the half-life of MLN in the periphery blood circulation is short and that the little amounts that remain in tissues provide a long-lasting effect.

Thus, MLN can be a preventive drug that blocks the entry, in vivo replication, and the spread of SARS-CoV-2 and diminishes inflammation, thus impacting severe COVID-19 morbidity. This application also shows the MLN effects on other viral diseases of public health importance, dengue, ZIKA, yellow fever, etc., and other non-viral pathogens that require the host's cell entry and exit, replication, and hyper-inflammation.

### Methods of Treatment

Given that infections by viruses, bacteria and protozoa involve cellular endocytosis and exocytosis pathways, targeting these pathways with chemotherapeutic agents offers the possibility of preventing or treating these infections (Glebov, 2020). Most bacteria and viruses, including SARS-CoV-2, utilize a clathrin-mediated endocytosis (CME) mechanism to gain access to the interior of cells (Grove & Marsh, 2011). The entry and egress of SARS-CoV-2 from host cells represent significant stages in COVID-19 pathogenesis, whereby CME is critical for infectivity and spread (Bayati et al., 2021). Exocytosis involves the release of SARS-CoV-2 to infect other cells.

The present application addresses the need for new therapeutic modalities for treating and preventing microbial infections, especially those of public health importance, as exemplified by RNA viruses associated with the current COVID-19 pandemic, as outbreaks of Dengue, Yellow fever, Zika, and Ebola infections.

In one aspect, the present application provides a method of preventing or treating a microbial infection in a subject, comprising administering a mycolactone composition in an amount effective for preventing or treating the infection.

In one embodiment, infection is caused by a virus.

In a more particular embodiment, the infection is caused by an RNA virus. In another embodiment, the infection is caused by a DNA virus. In another embodiment, the infection is caused by an enveloped DNA or RNA virus, especially an enveloped RNA virus.

In another embodiment, infection is caused by COV-19 virus.

In other embodiments, the infection is caused by a bacterium.

In other embodiments, the infection is caused by a fungus.

The methods of the present application may be applied to the prevention or treatment of variety of enveloped RNA and DNA viruses, including RNA viruses, such as retroviruses, lentiviruses, coronaviruses (including subgroup 1a and 1b alphacoronaviruses, subgroup 2a, 2b, 2c and 2d betacoronaviruses, and subgroup 3 gammacoronaviruses), herpesviruses, alphaviruses, bunyaviruses, filoviruses, flaviviruses, hepatitis viruses, orthomyxoviruses (e.g., influenza Types A, -B, -C, -D), paramyxoviruses, rhabdoviruses, and togaviruses; and DNA viruses, such as herpesviruses, poxviruses, and hepadnaviruses. Preferably, the microorganism or virus includes one or more cell surface proteins containing mannose residues. In certain preferred embodiments, the infection is caused by HIV, SARS-CoV-2, or an influenza Type 1 virus.

Exemplary species of enveloped viruses for prophylactic or therapeutic use, include retroviruses or lentiviruses, such as human immunodeficiency virus type 1 and type 2 (HIV-1 and HIV-2), human T-cell lymphotropic virus type I and type II (HTLV-I and HTLV-II); herpesviruses, including Epstein-Barr virus, human cytomegalovirus type 1 (HCMV-1), human herpes virus type 6 (HHV-6), human herpes virus type 7 (HHV-7), human herpes virus type 8 (HHV-8), influenza type A virus, including subtypes H1N1 and H5N1, as well as types -B, -C, and -D; coronaviruses, including severe acute respiratory syndrome coronavirus type 2 (SARS-CoV-2), SARS-CoV-1, Middle East Respiratory Syndrome Coronavirus (MERS-CoV), HCoV-229E, HCoV-OC43, HCoV-NL63, and HCoV-HKU1; RNA viruses that cause hemorrhagic fever, such as the filoviruses, Ebola virus (EBOV) and Marburg virus (MBGV); Bunyaviridae (e.g., Rift Valley fever virus (RVFV) and Crimean-Congo hemorrhagic fever virus (CCHFV)); and flaviviruses, such as Zika virus (ZIKV), Dengue fever virus (DENV), yellow fever virus (YFV), Hepatitis C virus, West Nile virus (WNV), tick-borne encephalitis virus, Saint Louis encephalitis virus, and GB virus C (GBV-C); enteroviruses (Types A to L, including coxsackieviruses (Types A to C); echoviruses; rhinoviruses (Types A to C), poliovirus); orthomyxoviruses (e.g., influenza Types A, -B, -C, -D, including A subtypes H1N1, H5N1, H3N2); paramyxoviruses (e.g., rubulavirus (mumps), rubeola virus (measles), respiratory syncytial virus, Newcastle disease, parainfluenza); parvoviruses (e.g., parvovirus B19 virus); rhabdoviruses (e.g., Rabies virus); arenaviruses (e.g., lymphocytic choriomeningitis virus and several Lassa fever viruses, including Guanarito virus, Junin virus, Lassa virus, Lujo virus, Machupo virus, Sabia virus, Whitewater Arroyo virus); alphaviruses (e.g., Venezuelan equine encephalitis virus, eastern equine encephalitis virus; western equine encephalitis virus); hepatitis A virus, hepatitis C virus (HCV), hepatitis D virus (HDV), hepatitis E virus (HEV), including any type, subtype, Glade or sub-Glade of the foregoing viruses.

In certain preferred embodiments, the RNA virus for prevention or treatment is a coronavirus, such as SARS-CoV-2, SARS-CoV-1, MERS-CoV, HCoV-229E, HCoV-OC43, HCoV-NL63, and HCoV-HKU1. In an exemplary embodiment, a method for preventing or reducing symptoms of a coronavirus infection, comprises orally administering to a subject in need thereof a composition comprising: an effective amount of an MLN composition; and at least one pharmaceutically acceptable carrier.

In other preferred embodiments, the RNA virus for prevention or treatment is a flavivirus, such as Zika virus (ZIKV), Dengue fever virus (DENY), yellow fever virus (YFV), Hepatitis C virus, West Nile virus (WNV), tick-borne encephalitis virus, Saint Louis encephalitis virus, and GB virus C (GBV-C). In an exemplary embodiment, a method for preventing or reducing symptoms of a flavivirus infection, comprises orally administering to a subject in need thereof a composition comprising: an effective amount of an MLN composition; and at least one pharmaceutically acceptable carrier.

In other preferred embodiments, the RNA virus for prevention or treatment is an influenza Type A virus. Influenza A viruses are divided into subtypes on the basis of two proteins on the surface of the virus, hemagglutinin (HA) and neuraminidase (NA). There are 18 known HA subtypes and 11 known NA subtypes. Many different combinations of HA and NA proteins are possible. For example, an “H7N2 virus” designates an influenza A virus subtype that has an HA7 protein and an NA2 protein. Similarly, an “H5N1” virus has an HA5 protein and an NA1 protein. Type A influenza viruses that may be targeted for prophylactic and/or therapeutic use according to the methods and compositions of the present application include a variety of sub-types, such as H1N1, H1N2, H3N2, H5N1, H5N2, H5N3, H5N4, H5N5, H5N6, H5N7, H5N8, and H5N9, H7N1, H7N2, H7N3, H7N4, H7N5, H7N6, H7N7, H7N8, H7N9, H9N1, H9N2, H9N3, H9N4, H9N5, H9N6, H9N7, H9N8, H9N9, H17N10 and H18N11).

In another exemplary embodiment, a method for preventing or reducing symptoms of an influenza Type A virus infection, comprises orally administering to a subject in need thereof a composition comprising: an effective amount of an MLN composition; and at least one pharmaceutically acceptable carrier.

Exemplary species of enveloped DNA viruses for prevention or treatment include, but are not limited to, Exemplary DNA viruses for prophylactic or therapeutic treatment include herpesviruses (e.g., HSV-1, HSV-2, EBV, VZV, HCMV-1, HHV-8), papillomaviruses (e.g., human papilloma virus (HPV) Types 1, 2, 4, 6, 11, 16, 18, 26, 30, 31, 33, 34, 35, 39, 40, 41, 42, 43, 44, 45, 51, 52, 54, 55, 56, 57, 58, 59, 61, 62, 64, 67, 68, 69, 70); poxviruses (e.g., smallpox virus), hepadnaviruses (Hepatitis B virus); anelloviruses (e.g., transfusion transmitted virus or torque teno virus (TTV); as well as any type, subtype, Glade or sub-Glade thereof.

In some embodiments, the MLN composition is used for the treatment or prevention of bacterial infection. Exemplary bacteria for treatment include, but are not limited to, Staphylococcus species, including S. epidermidis, S. aureus, and methicillin-resistant S. aureus; Enterococcus species, including E. faecalis and E. faecium; Mycobacterium tuberculosis, Haemophilus influenzae, Pseudomonas species, including P. aeruginosa, P. pseudomallei, and P. mallei; Salmonella species, including S. enterocolitis, S. typhimurium, S. enteritidis, S. bongori, and S. choleraesuis; Shigella species, including S. flexneri, S. sonnei, S. dysenteriae, and S. boydii; Brucella species, including B. melitensis, B. suis, B. abortus, and B. pertussis; Neisseria species, including N. meningitidis and N. gonorrhoeae; Escherichia coli, including enterotoxigenic E. coli (ETEC); Vibrio cholerae, Helicobacter pylori, Chlamydia trachomatis, Clostridium difficile, Cryptococcus neoformans, Moraxella catarrhalis, Campylobacter species, including C. jejuni; Corynebacterium species, including C. diphtheriae, C. ulcerans, C. pseudotuberculosis, C. pseudodiphtheriticum, C. urealyticum, C. hemolyticum, C. equi; Streptococcus species, including S. pneumoniae, S. pyogenes, S. mutans, S. agalactiae, S. equi, S. canis, S. bovis, S. equinus, S. anginosus, S. sanguis, S. salivarius, S. mills; Listeria monocytogenes, Nocardia asteroides, Bacteroides species, Actinomycetes species, Treponema pallidum, Leptospirosa species, Klebsiella pneumoniae; Proteus sp., including Proteus vulgaris; Serratia species, Acinetobacter, Yersinia species, including Y. pestis and Y. pseudotuberculosis; Francisella tularensis, Enterobacter species, Bacteriodes species, Legionella species, Borrelia burgdorferi, and the like.

In some embodiments, the MLN composition is used for the treatment or prevention of a fungal infection. Exemplary fungi for treatment include, but are not limited to, Aspergillus species, Dermatophytes, Blastomyces derinatitidis, Candida species, including C. albicans and C. krusei; Malassezia furfur, Exophiala werneckii, Piedraia hortai, Trichosporon beigelii, Pseudallescheria boydii, Madurella grisea, Histoplasma capsulatum, Sporothrix schenckii, Histoplasma capsulatum, Tinea species, including T. versicolor, T. pedis, T. unguium, T. cruris, T. capitus, T. corporis, T. barbae; Trichophyton species, including T. rubrum, T. interdigitale, T. tonsurans, T. violaceum, T. yaoundei, T. schoenleinii, T. megninii, T. soudanense, T. equinum, T. erinacei, and T. verrucosum; Microsporum species, including M. audouini, M. ferrugineum, M. canis, M. nanum, M. distortum, M. gypseum, M. fulvum, and the like.

In certain embodiments, the MLN composition is used for the treatment of pain and inflammation. In particular embodiments, the MLN compositions is used as an analgesic. In particular embodiments, the MLN composition is used as an anti-inflammatory agent. In certain embodiments, the MLN composition is used in providing systemic protection against chronic skin inflammation and inflammatory pain. In specific embodiments, the MLN composition is used as an adjuvant in treatment of cancer or heart disease, where relief of pain or inflammantion is needed. In certain embodiments, the inflammatory diseases treated by the MLB composition include one or more of Alzheimer's, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease and ulcerative colitis.

### Route and Dose of MLN Composition Administration

The MLN composition of the present application may be administered orally, intrathecally, intra-arterially, intravenously, intradermally, subcutaneously, transdermally (topically) or transmucosally. The MLN composition may be administered by any route, including oral, rectal, pulmonary, sublingual, and parenteral administration. Parenteral administration includes, for example, intraperitoneal, intravenous, intramuscular, intraarterial, intravesical (e.g., to the bladder), intradermal, transdermal, topical, or subcutaneous administration.

As a general proposition, the therapeutically effective amount of the MLN composition administered will be in a weight range of about 1 ng/kg body weight/day to about 100 mg/kg body weight/day whether by one or more administrations. In more particular embodiments, the MLN composition is administered in weight range from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In other embodiments, the MLN composition is administered at a dosage range of 1 ng-10 ng per injection, 10 ng-100 ng per injection, 100 ng-1 μg per injection, 1 μg-10 μg per injection, 10 μg-100 μg per injection, 100 μg-1 mg per injection, 1 mg-10 mg per injection, 10 mg-100 mg per injection, and 100 mg-1000 mg per injection. The MLN composition may be injected once daily, twice daily, three times daily, and/or every 2, 3, 4, 5, 6 or 7 days. In addition, the MLN composition may be administered over a period of one month, two months, six months, 12 months, 2 years, 5 years, 10 years, 20 years, or more.

In other embodiments, the MLN composition may be administered in a range from about 1 ng/kg to about 100 mg/kg. In more particular embodiments, the MLN composition may be administered in a range from about 1 ng/kg to about 10 ng/kg, about 10 ng/kg to about 100 ng/kg, about 100 ng/kg to about 1 μg/kg, about 1 μg/kg to about 10 μg/kg, about 10 μg/kg to about 100 μg/kg, about 100 μg/kg to about 1 mg/kg, about 1 mg/kg to about 10 mg/kg, about 10 mg/kg to about 100 mg/kg, about 0.5 mg/kg to about 30 mg/kg, and about 1 mg/kg to about 15 mg/kg.

In other particular embodiments, the amount of the MLN composition administered is, or is about, 0.0006, 0.001, 0.003, 0.006, 0.01, 0.03, 0.06, 0.1, 0.3, 0.6, 1, 3, 6, 10, 30, 60, 100, 300, 600 and 1000 mg/day.

The specific dose of the MLN composition may be determined based on the particular circumstances of the individual patient including the size, weight, age and sex of the patient, the nature and stage of the disease, the aggressiveness of the disease, and the route of administration of the MLN composition.

In certain embodiments, the MLN composition may be administered at least once per day, typically once, twice, three times or four times per day with the doses given at equal intervals throughout the day and night to maintain a constant presence of the MLN compound to provide sufficient antimicrobial activity. However, a skilled artisan will appreciate that a treatment schedule can be optimized for any given patient, and that administration of compound may occur less frequently than once per day.

In other embodiments, MLN composition of the present application is prescribed to be taken in combination with other antimicrobial and/or the other active agents. When used in such combinations, the MLN composition of the present application and other antimicrobial agents may be administered simultaneously, by the same or different routes, or at various times during treatment.

The treatment may be carried out for as long a period as necessary, i.e., until the infection is cleared or no longer a threat to the host. In some cases, the treatment may be continued indefinitely while the disease state persists, although discontinuation might be indicated if the antimicrobial compositions no longer produce a beneficial effect. In one embodiment, the treatment is carried out for 6 months and then discontinued. The treating physician can determine whether to increase, decrease, or interrupt treatment based on a patient's response, including evaluation of immune responses, viral loads etc.

### Pharmaceutical Compositions

As used herein the language “pharmaceutically acceptable carrier” is intended to include all solvents, solubilizers, fillers, stabilizers, binders, absorbents, bases, buffering agents, lubricants, controlled release, vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except as far as any conventional media or agent is incompatible with the active compound, use thereof in the compositions is contemplated. Supplementary agents can also be incorporated into the compositions. In certain embodiments, the pharmaceutically acceptable carrier comprises serum albumin.

The MLN pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene, glycol, and liquid polyethylene glycol, and the like), and suitable mixtures thereof. The proper fluidity can be maintained, for example, using a coating such as lecithin, by the maintenance of the required particle size in the case of dispersion and using surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating an active agent in the required amount in an appropriate solvent with one or a combination of ingredients enumerated above, as required, followed by filtered sterilization. Dispersions can be prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral therapeutic administration, the active compound may be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished using nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and poly lactic acid. Methods for preparation of such formulations will be apparent to those skilled in the art. The materials can also be obtained commercially from e.g., Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Suitable unit dosage forms include, but are not limited to powders, tablets, pills, capsules, lozenges, suppositories, patches, nasal sprays, injectibles, implantable sustained-release formulations, lipid complexes, etc.

A dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the present application is dictated by and directly dependent on the unique characteristics of the active compound and the particular therapeutic effect to be achieved, and the limitations inherent in the art of compounding such an active compound for the treatment of individuals.

Toxicity and therapeutic efficacy of the MLN composition of the present application can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. MLN compounds exhibiting large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to determine useful doses more accurately in humans. The pharmaceutical compositions can be included in a container, pack, or dispenser together with instructions for administration.

The present application is further illustrated by the following examples that should not be construed as limiting. The contents of all references, patents, and published patent applications cited throughout this application, as well as the Figures and Tables, are incorporated herein by reference.

### EXAMPLES

### Materials and Methods

### MLN and Exocytosis

The detailed computational methods employed to investigate the inhibition of exocytosis in platelets and mast cells are known in the art. Briefly, molecular dockings of MLN to proteins were performed, and molecular mechanics Poisson-Boltzmann surface area (MM-PBSA) binding energy calculations of MLN and Munc18b complex were done with 100 ns molecular dynamics simulations. The target proteins were the soluble n-ethylmaleimide-sensitive factor attachment protein receptor (SNARE), the vesicle associated membrane protein 8 (VAMPS), synaptosomal-associated protein (SNAP23), syntaxin 11, Munc13-4 in mast cells (its isoform Munc13-1 was used), and Munc18b, and other published known MLN targets sec61, angiotensin II type 2 receptor (AT2R), and Wiskott-Aldrich Syndrome protein (WASP).

### MLN and Endocytosis

### Protein Structure Retrieval and Preprocessing

The experimentally solved three-dimensional (3D) structure of the clathrin-terminal domain (CTD) was retrieved from the Protein Data Bank with accession number PDB ID: 2XZG. The removal of available water molecules and ligands was done in PyMOL v 4.0.0. The structure was then minimized in GROMACS v 2018 using the steepest descent algorithm at steps. GROMOS96 43a1 force field was used to generate the protein topology and position restrain files. Periodic Boundary Conditions (PBC) were applied to the structure with the protein centered 1 nm from the edge of a cubic box to monitor the movement of all particles and avoid edge effects on the surface atoms. The resulting structure was solvated with SPC water and neutralized with Na and Cl atoms.

### Molecular Docking of MLN to CTD

AutoDock Vina in PyRx was used to dock MLN against the minimized CTD protein structure. The .sdf format of MLN with ID: 5282079 was retrieved from Pub-Chem and imported into OpenBabel. It was then minimized using the Universal Force Field (UFF) for 200 steps and optimized using the conjugate gradient. A grid box of dimensions 63.589, 59.346, 53.464 Å and center 44.251, 44.1745, 44.3269 Å that covered the entire protein surface was set for docking. Additionally, a default exhaustiveness of eight was used.

### Molecular Dynamics Simulations of MLN-CTD Complexes

One hundred ns MD simulations of the MLN-CTD complex were performed using GROMACS v 2018. The protein topologies were initially generated using the GROMOS96 43a1 force field and the ligand topologies via the PRODRG server. A complex was formed by merging the topologies. The complex was then solvated with water molecules in a cubic box of size 1.0 nm3 and neutralized with Na and Cl ions. Energy minimization of the complex was conducted for 50,000 steps using the steepest descent algorithm. Mycolactone was restrained before the constant-temperature, constant-volume (NVT), and constant temperature, constant-pressure ensemble (NPT) simulation. Equilibration of each complex was performed for 100 ps apiece, and the final MD simulation was conducted for 100 ns with time steps of 2 fs under particle mesh Ewald (PME). The free binding energies were calculated using g mmpbsa. The binding free energy contribution per residue was calculated using MM-PB SA, and the output plots were generated with R.

### Reverse Target Searches for Novel Binding Pocket

The 3D model structures of both MLN A and B were predicted using the steepest descent algorithm with force field UFF, where all atoms move in Avogadro software v. 1.2. MLN predicted target pool was analyzed by a battery of servers in search of possible targets using TargetHunter, Pharmmapper, Spider, SuperPred, Stitch, Hitpick, reversescreen3D, and Swiss target prediction to compare this with the mutant viral spike proteins, Wild type (PDB id: 7TAT), Delta-Plus (PDB ID: 7W9E), and Omicron (PDB id: 7Q07) was docked with both MLN A and B using the glide module of Schrodinger software v. 2022-3 and XP scores were calculated. Further, the top-scoring complexes of wild type were subjected to 20 ns MD simulations.

### SARS-CoV-2 Strains and Cell Lines

Human alveolar basal epithelial A549-ACE2 cells and SARS-CoV-2 [novel coronavirus (nCoV)/Washington/1/2020] provided via the World Reference Center for Emerging Viruses and Arboviruses (Galveston, TX, USA) and from BEI Resources. Variants of concern were obtained from BEI resources. Delta Variant (BEI Cat.ID. NR-55671) Isolate hCoV-19/USA/MD-HP05285/2021 (Lineage B.1.617.2); Omicron Variant (BEI Cat.ID. NR-56461) Isolate hCoV-19/USA/1VDHP20874/2021 (Lineage B.1.1.529).

### Antiviral Inhibition Assays

### Evaluation of Viral Inhibition by MLN Using Spike Immunohistochemistry Assay

All SARS-CoV-2 infections were performed under biosafety level 3 conditions on the human cells in DMEM supplemented with 2% fetal bovine serum (FBS) and antibiotics. For the preliminary selection of hits, cells were pre-treated with MLN or other inhibitors for 2 h with 2-fold dilutions beginning at 50 μM in triplicate for each assay. To enumerate the IC50 or percent inhibition, an identical treatment was performed with 10-fold dilutions beginning at 50 μM (134.6 nM in the case of MLN). A549-ACE2 cells were infected with a multiplicity of infection (MOI) of 0.5 in media containing the appropriate concentration of drugs. After 48 h, the cells were fixed with 10% formalin, blocked, and probed with mouse anti-Spike antibody (GTX632604, GeneTex, Irvine, CA, USA) diluted 1:1000 for 4 h, rinsed, and probed with an anti-mouse-horseradish peroxidase (HRP) for 1 h, washed, then developed with 3, 30 Diaminobenzidine (DAB) substrate for 10 min. Spike-positive cells (n>40) were quantified by light microscopy as blinded samples.

### Evaluation of Viral Inhibition by MLN Using Plaque Assay

Viral titers were determined by plaque assay. Briefly, a monolayer of cells was infected with serial dilutions of virus samples for 1 h at 37 C. The viral inoculum is then removed and replaced by a MEM overlay media containing 1.25% carboxymethyl cellulose. Cells were incubated for 72 h, after which the overlay media was removed, and cells were fixed with 10% formalin and stained with 0.25% crystal violet solution. Plaques are then counted, and the viral concentration is calculated using the following method. The average value of plaques in replicate wells x dilution factor÷virus inoculum volume (in mL)=titer in PFU/mL. The data were analyzed and plotted using GraphPad Prism v. 9.5.1. (GraphPad Software Inc., San Diego, CA, USA), and IC50 values were extrapolated from the nonlinear fit of the response curves.

### MLN and Virus Entry and Spread Inhibition Assay

A549-ACE2 cells were treated with 134.6 nM MLN (-100-fold higher than IC50) 2 h before infection with SARS-CoV-2 and 2 h after infection with SARS-CoV-2 with a protocol modified from Chianese et al., 2022. This experiment assessed whether MLN would block the entry of the virus to cells and, if the virus gets infected, whether it blocks the re-entry/spread to neighboring cells. Cells were infected with an MOI of 0.5 for 2 h. Then, the infection medium was replaced with a medium containing MLN or dimethyl sulfoxide (DMSO as vehicle control), and the samples were incubated at 37 C for 24 h. The plates were probed by mouse anti-Spike antibody (GTX632604, GeneTex, Irvine, CA, USA) and read.

### Cytotoxicity Assays

To confirm if the MLN has no adverse effect on the host cells, we conducted cytotoxicity tests on various cell lines, primarily the lung epithelium, kidney, and liver, i.e., A549, HEK293, and HUH7.1 cell lines. MLN cytotoxicity/cytostatic effect against different cell lines could only be tested at a maximum concentration of 1.34 μM again due to a dilute stock source in 96-well format. A549, HEK293, and HUH7 cells were maintained in filter cap cell culture flasks at 37 C in a humidified atmosphere containing 5% CO2 and Dulbecco's modified Eagle's media supplemented with 10% fetal bovine serum and 1% penicillin-streptomycin (Gibco Life Technologies, Cergy-Pontoise, France). A549, HEK293, and HUH7 cells were seeded in separate plates in a 96-well black clear bottom cell culture grade plate at a density of 5000 trypsinized cells/100 L/well; cell counting was performed by trypan blue (sigma) live cell staining and automated counting by Invitrogen Countess 3 automated cell counter. After adding various test drugs/compounds to the test plates, Amphotericin B as a positive control and DMSO vehicle (same as sample volume 1 L as the negative control), the plates were incubated for 24 h at 37 C and humidified at 5% CO2. Compounds were dissolved in cell culture-grade DMSO (stock concentration: 10 mM; highest concentration: 100 mM; 100× diluted for other compounds and 1.34 μM for MLN). The highest concentrations were serially diluted by a factor of 2:1 10 times, with each series being carried out in triplicates. The Hoechst 33,342 dye from Thermo Fisher, Waltham, MA USA was used to stain the incubated plates, which were then incubated for 24 h at 37 C with 5% CO2 and humid conditions. The cells were then imaged using a 4× plan fluor objective (4× Plan Apo Lambda Nikon air objective lens with a camera binning of 2 and a pixel size of 3.367 m×3.367 m) with bright field and LED illumination capture DAPI channels by Sony CMOS inbuilt camera on the ImageXpress Pico High-Content Imaging System Microscope from Molecular Devices, San Jose, CA, USA. Additionally, automated data processing tools with pre-configured analysis algorithms were used to process the imaging data, and nucleus counts were recorded. GraphPad prism software version 9.5.1 was used to perform Nonlinear regression (curve) utilizing normalized values on the Y-axis and log transformed drug concentrations on the X-axis to quantify cell death and determine CC50 of test compounds. The maximum drug concentration effect on cells was used to make the graph for maximum toxicity observed, and corresponding ratios with average IC50s against different strains were calculated.

### Example 1. In Silico Binding of MLN to a Viral Protein Associated With Virus-Mediated Exocytosis

Mycolactone (MLN), a macrolide toxin produced by Mycobacterium ulcerans, was shown to impair granule exocytosis in red blood platelets (RBPs) and mast cells of infected individuals by binding a SNARE protein, specifically the syntaxin chaperone protein, Munc18b. Mycolactone is the causative agent of Buruli ulcer and is known to arrest inflammatory processes that would otherwise initiate wound healing processes in Buruli ulcer (BU) patients. The main symptom in infected patients is the development of painless skin ulcers that over time become extensive requiring palliative surgery. The net result of this is that the contents of the RBPs' alpha and the small granules do not exit into the periphery blood to initiate the cascading processes of wound healing.

Exocytosis and endocytosis are vital processes in viral infections. Most bacteria and viruses, such as SARS-CoV-2, use the clathrin-mediated endocytosis (CME) pathway to gain access to the interior of cells. As noted above, endocytosis and exocytosis of SARS-CoV-2 from host cells represent significant stages in COVID-19 pathogenesis, whereby CME is critical for entry (infectivity) and spread.

In accordance with the present application, computational methods were carried out in silico to evaluate the ability of MLN to inhibit viral endocytosis. In this case, the clathrin terminal domain (CTD) was targeted, since it serves as the center for protein-protein interactions, whereby its inhibition was reported to interfere with clathrin-mediated endocytosis. Using the MM-PB SA calculations, the average free binding energy was −59.210 kJ/mol for the mycolactone-CTD complex. The contributory electrostatic, polar, non-polar, and van der Waals energies were −7.388 kJ/mol, 31.705 kJ/mol, −7.463 kJ/mol, and −76.177 kJ/mol, respectively. Additionally, a per-residue decomposition of the binding energy revealed Met99 contributed significant energies of −5.2131 kJ/mol.

In this study, MLN docked firmly inside the binding pocket (FIG. 1a) with a low binding energy of −9.0 kcal/mol. It also interacted with the CTD protein (FIG. 1b) via hydrophobic interactions with Ala160, Ala202, Glu212, Gln203, Asp271, Glu268, Phe204, Glu207, Pro308, Lys269, Ser267, Arg354, Leu357, Va1353 and hydrogen bonding with Arg157 [3.08 Å], Thr158 [3.05 Å], Phe210 [3.02 Å, 2.85 Å], Ile226 [3.32 Å], and Ser200 [2.74 Å, 2.90].

The results of this analysis showed that MLN strongly binds to the CTD associated with clathrin-mediated endocytosis (CME) in mammalian host cells. Additionally, reverse target searches for this potential lead were performed to identify targets within the viral proteome. More particular, the use of Swiss target hunter and Pharmmapper servers strongly suggested a specific interaction between MLN and heptad repeat (HR) region domains present in viral fusion proteins.

HRs are present in a number of viruses, especially enveloped viruses, such as SARS-CoV-2, which is known to contain HR1 and HR2 regions (forming the basal S2 domain) that form a hetero hexamer fusing the viral envelope to the target cell viral membrane or with the endosome membrane delivering nucleocapsid to the cytoplasm. This region constitutes the bottleneck for viral infections as viral entry into the cell is the most critical step.

A 100 ns MD simulation was performed to understand the structural stability and

conformational changes when situated under dynamic physiological conditions [38]. The parameters evaluated were the root mean square deviation (RMSD), the radius of gyration (Rg), and the root means square fluctuation (RMSF). The RMSD is a plausible measure of protein stability that accesses the deviation of the protein-ligand complex during the simulation from the initial protein backbone atomic coordinates [39]. The protein main-tained an average RMSD of 0.3 nm (FIG. 2a), suggesting the stability of the structure. Considering the RMSF, sizable fluctuations were observed at numerous residue positions (FIG. 2b). Furthermore, the protein was stably folded per its Rg plot (FIG. 2c). The Rg decreased during the first 40 ns and remained stable for the rest of the simulation time. The binding free energies of the complexes were estimated using MM-PBSA calculations. The calculations address some limitations of current scoring functions [40]. An average free binding energy of −59.210 kJ/mol was computed for the MLN-CTD complex. Energy terms, namely electrostatic, polar, non-polar, and van der Waals, contributed energies of −7.388 kJ/mol, 31.705 kJ/mol, −7.463 kJ/mol, and −76.177 kJ/mol, respectively, to the free binding energy. Additionally, a per-residue decomposition of the binding energy revealed that Met99 contributed significant energy of −5.2131 kJ/mol.

### Example 2. MLN Metabolites Bind to the HR1 Domain of Viral Fusion Proteins

Docking and MD simulations strongly supports the interaction between MLN and the SARS-CoV-2 spike protein. The major metabolites of MLN are MLN-A and MLN-B. The 3D models of virus binding to MLN A & B were predicted using the steepest descent algorithm with force field UFF, where all atoms move in Avogadro software. MLN predicted target pool was analyzed by a battery of servers in search of possible targets like TargetHunter, Pharmmapper, Spider, SuperPred, Stitch, Hitpick, reversescreen3D, and Swiss target prediction. Both Pharmmapper and Swiss target servers suggested the viral fusion domains (VFDs) as one of the top possible hits. The basis of these consensus predictions was reported with the binding of highly pharmaco-similar compounds to various VFDs.

The availability of high-resolution structures of spike proteins of SARS-CoV-2 the interaction studies were helpful in enabling these studies. However, to stabilize crystal structures, the SARS-CoV-2 HR2 domain of the spike protein was truncated to avoid VFD activation and structural distortion. The heptad repeat region one of S2 domain (HR1) was intact and used to perform docking and simulation studies.

Both Pharmmapper and Swiss target servers suggested the viral fusion domain (VFD) as one of the possible MLN top hits. The basis of these consensus predictions was reported with the binding of highly pharmaco-similar compounds to various VFDs. With the availability of high-resolution structures of spike proteins of SARS-CoV-2, the inter-action studies were simple (FIG. 3a,b). However, to stabilize crystal structures HR2 domain was truncated to avoid VFD activation and structural distortion. The heptad repeat region of the S2 domain (HR1) was intact and used to perform docking and simulation studies. Furthermore, the top-scoring complexes of wild type were subjected to 20 ns MD simulations.

The results of these analyses showed that MLN-A and MLN-B were both found to specifically bind to the SARS-CoV-2 HR1 region in the spike protein, which dimerizes with HR2 upon activation.

To compare the binding to mutant viral spike proteins, Wild type (PDB id: 7TAT), Delta-Plus (PDB id: 7W9E), and Omicron (PDB id: 7Q07) strains were docked with both MLN A& B using the glide module of Schrodinger software (Schrodinger, 2019) and XP scores were calculated. Further, the top-scoring complexes of wild type were subjected to 20 ns MD simulations. The findings of these analyses show that the docking interactions of MLN with the three spike proteins were highly consistent owing to the highly conserved sequence and functional importance to the viral infection cycle. The MD simulation validated the high energy docking (dG_{bind}=−62.518) and the complex was highly stable throughout 20 ns MD simulations with the whole spike involved in the interaction.

Given that the same endocytosis pathway is utilized in several enveloped viruses, including HIV and Zika, MLN and derivatives thereof serve as potent compounds for blocking endocytosis, exocytosis, and replication in mammalian cells by many viruses. The in-silico modeling established the potential ability of MLN to serve as a candidate drug for interfering with endocytosis, exocytosis, and viral replication of SARS-CoV-2 in mammalian cells.

### Example 3. MLN Inhibits Live SARS-CoV-2 Infections

To confirm the in-silico findings experimentally, a live SARS-CoV-2 viral assays were performed in the presence and absence of MLN. In these experiments, SARS-CoV-2 infections were performed in biosafety level 3 conditions on human A549-ACE2 cells in DMEM +2% FBS.

In one assay, viral inhibition of test compounds was carried out by a Spike Immunohistochemistry (IHC) assay. For the preliminary selection of hits, cells were pre-treated with Mycolactone (MLN) or other inhibitors for 2 hours with 2-fold dilutions beginning at 50 μM in triplicate for each assay. To enumerate the ICso or percent inhibition, an identical treatment was performed with 10-fold dilutions beginning at 50 μM.

The A549-ACE2 cells were infected with an MOI (multiplicity of infection) of 0.5 in media containing the appropriate concentration of drugs. Cells were infected at an MOI of 0.1 in media containing the appropriate concentration of drugs. After 48 hours, the cells were fixed with 10% formalin, blocked, and probed with mouse anti-Spike antibody (GTX632604, GeneTex) diluted 1:1,000 for 4 hours, rinsed, and probed with an anti-mouse-HRP for 1 hour, washed, then developed with DAB substrate 10 minutes. Spike positive cells (n>40) were quantified by light microscopy as blinded samples.

SARS-CoV-2 inhibition by test compounds and their comparison is described in Table 1. The IC50 of MLN vs. Remdesivir (positive control) and DMSO (negative control) is described in FIG. 5.

TABLE 1
Comparative potency breakpoints of leading antiSARS-CoV2
in comparison with MLN (0.269 mM stock).

| | IC-50, | 80% Inhibition of |
|---|---|---|
| Name | Anti-SARS-CoV-2 (μM) | SARS-CoV-2 (μM) |
| β-D-N4- | 0.10 ± 0.06 | 0.30 ± 0.09 |
| hydroxycytidine | | |
| (molnupiravir) | | |
| PF-00835231 (Pfizer) | 0.23 ± 0.03 (with P-gp | 0.68 ± 0.023 |
| | efflux inhibitor) | |
| MLN | 0.02 ± 0.006 | 0.03 ± 0.005 |

The results from the IHC assay (in triplicate) established that MLN is highly potent against SARS-CoV-2, exhibiting 100% viral inhibition against SARS-CoV-2 when used up to 60nM and an average of 84% inhibition at 30nM.

To further substantiate the results from the IHC assay, plaque assays were carried out to further evaluate inhibition of SARS-CoV-2 variants of concern (VOC) by MLN and Remdesivir, an FDA-approved drug for SARS-CoV-2. Viral titers were determined by plaque assay. Briefly, a monolayer of cells is infected with a series of serial dilutions of virus samples for 1 hour at 37° C. The viral inoculum was then removed and replaced with an MEM overlay media containing 1.25% carboxymethyl cellulose. Cells were then incubated for 72 hours after which the overlay media was removed, and the cells were fixed with 10% formalin and stained with 0.25% crystal violet solution. Plaques are counted in the dilution wells containing between 10-100 plaques so that the original concentration of the viral sample could be calculated. Data were analyzed and plotted using GraphPad Prism and EC50 values were extracted from the nonlinear fit of response curves. As shown in FIG. 6, the results from the plaque assay showed that MLN exhibited IC50 values between about 9-18-fold lower than Remdesivir.

To further evaluate the inhibition of SARS-CoV-2 infection by MLN, a viral entry and spread inhibition assay was performed to assess whether MLN blocks entry of virus into cells and, if the virus gets infected, whether it blocks the re-entry/spread to neighboring cells. Briefly, A549-ACE2 cells were treated with 5pM MLN (100-fold higher than IC-50) either 2 hours before infection with SARS-CoV-2 or 2 hours after infection with SARS-CoV-2. The cells were infected at an MOI of 0.5 for 2 hours. Then, the infection medium was replaced with a medium containing MLN or dimethyl sulfoxide (DMSO as vehicle control), and the samples were incubated at 37° C. for 24 hours. Infected wells were then analyzed by Spike IHC and Plaque Assay. The results are illustrated in FIG. 6, which shows that MLN demonstrated consistent, complete blocking activity against the entry and spread of SARS-CoV-2.

### Example 4. Lack of Toxicity in Host Cells at Effective MLN Concentrations

To determine if MLN exerts adverse effects on the host cells, cytotoxicity experiments were carried out on various cell lines representing primarily the kidney and liver, i.e., A549, HEK293 and HUH7.1 cells. In these experiments, MLN cytotoxicity/cytostatic effects against the different cell lines could only be tested at a maximum concentration of 1.34 μM due to the diluent stock in the 96-well format. MLN exhibited high variability in toxicity among the different cell lines. The cytotoxicity of MLN against Human alveolar cell line—A549 was 17.12±9.1% (FIG. 7). Cytotoxicity against immortalized Human fetal renal cell line HEK293 was 40.30±3.6%. [C] Cytotoxicity against Human hepatoma cell line Huh7.1 was 36.25±5.6% (FIG. 7). This makes cytotoxicity IC50 breakpoint ratio versus anti-SARS-CoV-2 activity more than 65×.

The in-silico data and highly specific activity of MLN demonstrate that MLN is a spike fusion blocker targeting a highly conserved region among different SARS-CoV-2 strains such that its activity doesn't vary in spite of the high turnover rate of SARS-CoV-2 VOCs.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended unless the context specifically indicates the contrary.

What is claimed is:

1. A method of treating a microbial infection in a subject, comprising administering to a subject in need thereof an effective amount of a composition comprising mycolactone (MLN).

2. The method of claim 1, wherein the subject is infected with a virus.

3. The method of claim 2, wherein the subject is infected with an enveloped virus.

4. The method of claim 2, wherein the subject is infected with an RNA virus.

4. hod of claim 4, wherein the RNA virus is a coronavirus.

6. The method of claim 5, wherein the coronavirus is SARS-CoV-2, SARS-CoV-1, MERS-CoV, HCoV-229E, HCoV-OC43, HCoV-NL63, or HCoV-HKU1.

7. The method of claim 6, wherein the coronavirus is SARS-CoV-2.

8. The method of claim 4, wherein the RNA virus is a flavivirus.

9. The method of claim 8, wherein the flavivirus is Zika virus, Dengue fever virus, yellow fever virus, Hepatitis C virus, West Nile virus, tick-borne encephalitis virus, Saint Louis encephalitis virus, or GB virus C.

10. The method of claim 4, wherein the RNA virus is a Filovirus.

11. The method of claim 10, wherein the flavivirus is Ebola virus

12. The method of claim 1, wherein the subject is infected with a bacterium.

13. The method of claim 1, wherein the subject is infected with a fungus.

14. A method of preventing a microbial infection in a subject, comprising administering to a subject in need thereof an effective amount of a composition comprising mycolactone (MLN).

15. The method of claim 14, wherein the subject is infected with a virus.

16. The method of claim 15, wherein the subject is infected with an enveloped virus.

17. The method of claim 15, wherein the subject is infected with an RNA virus.

18. The method of claim 17, wherein the RNA virus is a coronavirus.

19. The method of claim 18, wherein the coronavirus is SARS-CoV-2, SARS-CoV-1, MERS-CoV, HCoV-229E, HCoV-OC43, HCoV-NL63, or HCoV-HKU1.

20. The method of claim 19, wherein the coronavirus is SARS-CoV-2.
